(19) United States
(12) Patent Application Publication
(10) Pub. No.: US 2006/0211631 A1
(43) Pub. Date: Sep. 21, 2006

(54) Transfusion preparation for peripheral intravenous administration and method of stabilizing vitamin b1

(76) Inventors: Yasuhiro Mitsumoto, Tokushima (JP); Shigeaki Arita, Tokushima (JP); Seiji Tani, Tokushima (JP); Nobuaki Sumiyoshi, Tokushima (JP)

Correspondence Address: SUGHRUE MION, PLLC, 2100 PENNSYLVANIA AVENUE, N.W., SUITE 800, WASHINGTON, DC, 20037, US

(21) Appl. No.: 10/556,772

(22) PCT Filed: May 14, 2004
(86) PCT No.: PCT/JP04/06539

§ 371 Date: Nov. 14, 2005

(30) Foreign Application Priority Data
May 22, 2003 (JP) 2003-145353

(51) Int. Cl. A61K 31/70; A61K 31/4172; A61K 31/405; A61K 31/401; A61K 31/198; A61K 31/51

(52) U.S. Cl. 514/23; 514/561; 514/562; 514/563; 514/564; 514/565; 514/566; 514/567; 514/400; 514/419; 514/423; 514/276

(57) ABSTRACT

The infusion preparation for peripheral parenteral administration comprises an infusion (A) containing glucose with 80 to 200 g/L concentration, and an infusion (B) containing amino acid with 50 to 150 g/L concentration. The infusion (A) and the infusion (B) are separately put into a vessel that is divided with an openable partition means. The infusion (A) does not contain sulfite, has titratable acidity of 1 or less, contains vitamin B1 and is adjusted to pH 3 to 5. The infusion (B) is adjusted to pH 6.5 to 8. After mixing the both infusions, the mixture has pH 6 to 7.5 and titratable acidity of 5 to 10.

### TECHNICAL FIELD

The present invention relates to an infusion preparation for peripheral parenteral administration that stably contains vitamin B1 and to a method for stabilizing vitamin B1.

### BACKGROUND ART

Conventionally, in order to supply all the nutrients necessary for life support to patients who have difficulties in receiving nourishment per oral, infusion therapy to administer an infusion through veins has been widely carried out. Nutrients to be administered include not only sugars, amino acids, electrolytes, but also minerals, vitamins and other nutrients necessary for life support.

It is well known that when a high-calorie infusion is administered through central veins (TPN, IVH), vitamin B1 deficiency inhibits aerobic glycolysis, thus producing lactic acid and causing severe lactic acidosis. Adding vitamin B1 is essential. For this reason, a high-calorie infusion preparation that has vitamin B1 added in advance has been under consideration (see Patent documents 1 to 4 below).

On the other hand, it is reported that even when a medium-calorie general infusion of nutrient is administered through peripheral veins during a relatively short period (PPN), vitamin B1 deficiency can occur by lowering vitamin B1 concentration in blood, though it is not so severe as to administer a high-calorie infusion (see Nonpatent document 1 below). Attempts have been made to add vitamin B1 also to an infusion for peripheral parenteral administration in advance (see Patent document 5).

Vitamin B1 solution is unstable between neutrality and alkaline, and is decomposed by sulfite ions. Therefore, by giving a specific pH to an infusion containing vitamin solution and adding no sulfite or minimum sulfite, vitamin B1 in the above infusion preparations has been stabilized.

In the above conventional infusion preparations, the infusion to which vitamin B1 is added has a specific characteristic, so that vitamin B1 can keep stabilized. Even so, there is still demand for further stabilized vitamin B1.

Patent document 1: Japanese Patent Publication Laid-Open No. 8-143459

Patent document 2: Japanese Patent Publication Laid-Open No. 9-59150

Patent document 3: Japanese Patent Publication Laid-Open No. 10-226636

Patent document 4: Japanese Patent Publication Laid-Open No. 11-35471

Patent document 5: Japanese Patent Publication Laid-Open No. 2003-55195

Nonpatent document 1: Nakamura et al., “Thiamine Deficiency in Critically Ill Patients under Peripheral Parenteral Nutrition.”, The Japanese Journal of Surgical Metabolism and Nutrition, 36 (6), 307 (2002)

### DISCLOSURE OF THE INVENTION

### Problem to be Solved by the Invention

The advantage of the present invention is to provide an infusion preparation for peripheral parenteral administration that contains much more stable vitamin B1 without losing safety and efficacy as a preparation, and a method for stabilizing vitamin B1.

### Means for Solving the Problem

After keen examinations to solve the above problem, the inventors of the present invention have found that buffering salt contained in an infusion prevents vitamin B1 from being stabilized. The inventors have achieved the present invention, finding the new fact that when electrolytes in a vitamin B1-containing infusion have titratable acidity of one or less, a preparation that contains much more stable vitamin B1 without losing safety and efficacy can be attained.

The infusion preparation for peripheral parenteral administration in the present invention comprises an infusion (A) containing glucose with 80 to 200 g/L concentration, and an infusion (B) containing amino acid with 50 to 150 g/L concentration. The infusion (A) and the infusion (B) are separately put into a vessel that is divided with an openable partition means. The infusion (A) does not contain sulfite, has titratable acidity of 1 or less, contains vitamin B1 and is adjusted to pH 3 to 5. The infusion (B) is adjusted to pH 6.5 to 8. After mixing the infusion (A) and the infusion (B), the mixture has pH 6 to 7.5 and titratable acidity of 5 to 10.

In the present invention, it is preferable that the infusion (A) contains carboxylic acid and its salt with 0 to 5 mEq/L concentration.

In the present invention, it is preferable that electrolytes contained in the infusion (A) are all strong electrolytes. The infusion (A) of the present invention may have electrolyte composition as follows: K⁺: 10 to 20 mEq/L, Ca²⁺: 2 to 10 mEq/L, Mg²⁺: 2 to 10 mEq/L, Cl⁻: 12 to 30 mEq/L, Zn: 2 to 10 mmol/L. The infusion (B) may have electrolyte composition as follows: Na⁺: 80 to 150 mEq/L, K⁺: 20 to 40 mEq/L, P: 10 to 20 mmol/L. The volume ratio (A:B) of the infusion (A) and the infusion (B) may be 1 to 4:1.

In particular, in the present invention, it is preferable that calcium and potassium are respectively contained in the form of chloride as the above strong electrolytes, and that sodium chloride is not used as a sodium supply source in either of the infusions (A) or (B).

Moreover, preferably, the vessel is a flexible plastic infusion bag having at least two chambers that are separated by an easily removable seal.

According to the method for stabilizing vitamin B1 in the present invention, in an infusion preparation for peripheral parenteral administration which comprises an infusion (A) containing vitamin B1 and 80 to 200 g/L of glucose and an infusion (B) containing 50 to 150 g/L of amino acid and being adjusted to pH 6.5 to 8 that are separately put into a vessel that is divided with an openable partition means and wherein after mixing the infusion (A) and the infusion (B), the mixture has pH 6 to 7.5 and titratable acidity of 5 to 10, the infusion (A) does not contain sulfite, has titratable acidity of 1 or less and is adjusted to pH 3 to 5.

In this method for stabilizing vitamin B1, the infusion (A) may contain carboxylic acid and its salt with 0 to 5 mEq/L concentration, and electrolytes contained in the infusion (A) may be all strong electrolytes.

### EFFECT OF THE INVENTION

The infusion preparation for peripheral parenteral administration in the present invention has an effect of further improving stability of vitamin B1 without losing safety and efficacy.

### PREFERRED EMBODIMENTS FOR PRACTICING THE INVENTION

The infusion preparation for peripheral parenteral administration (hereafter, referred to simply as infusion preparation) in the present invention will be now described in detail. In the infusion preparation of the present invention, the infusion (A) containing glucose and the infusion (B) containing amino acid are separately put into a vessel that is divided with an openable partition means, and when used, the infusions (A) and (B) are mixed.

<Infusion (A)>

In the present invention, the infusion (A) is basically composed of glucose, strong electrolytes and vitamin B1. To stabilize vitamin B1, the infusion (A) does not contain sulfite. Glucose is used with 80 to 200 μL concentration, preferably, with 80 to 150 g/L concentration. In stabilizing vitamin B1, it is more preferable to control the concentration of carboxylic acid and its salt to 0 to 5 mEq/L in this glucose solution. It is also preferable that pH in the glucose solution is adjusted with mineral acid such as hydrochloric acid to eliminate buffer property as much as possible and that the electrolytes contained in the glucose solution are all strong electrolytes. The pH range of the infusion (A) is 3 to 5, preferably, 3.5 to 4.5. The infusion (A) at pH of not more than 3 can be excellent in stabilizing vitamin B1 while allowing glucose to be unstable. On the other hand, the infusion (A) at pH of not less than 5 allows vitamin B1 to lose stability.

Preferably, the glucose solution has fluid volume of 200 to 100 mL. Distilled water for injection is normally used as a solvent of the glucose solution. In addition, to further improve stability of vitamin B1 and make it easy to adjust the pH of the mixture described later to 6 to 7.5, the glucose solution has titratable acidity of 1 or less, preferably 0.5 or less, more preferably 0.1 or less.

Besides glucose, one or more of reducing sugars such as fructose, maltose etc., or nonreducing sugars such as sorbitol, glycerin etc. may be contained in moderation.

<Infusion (B)>

In the present invention, the infusion (B) is an amino acid solution, and contains amino acid composition composed of at least essential amino acid. Amino acid is contained with 50 to 150 g/L concentration, preferably, with 80 to 120 g/L concentration, in terms of free amino acid. Each amino acid used is preferably purely crystalline amino acid as in a general amino acid infusion. Though these amino acids are normally used in the form of free amino acid, they can be used not only in such a form, but also in the form of pharmacologically accepted salt, ester, N-acyl derivative, salt of two amino acids and peptide. Especially, it is suitable that L-cysteine is contained as N-acetyl compound in terms of stability. Fluid volume of the infusion (B) to be put into an infusion vessel may be 100 to 500 mL. Distilled water for injection is normally used as a solvent of the infusion (B).

Preferable composition of amino acid is shown as follows in terms of free amino acid: L-leucine: 10 to 20 (g/L), L-isoleucine: 5 to 15 (g/L), L-valine: 5 to 15 (g/L), L-lysine: 5 to 15 (g/L), L-threonine: 2 to 10 (g/L), L-tryptophan: 0.5 to 5 (g/L), L-methionine: 1 to 8 (g/L), L-phenylalanine: 3 to 15 (g/L), L-cysteine: 0.1 to 3 (g/L), L-tyrosine: 0.1 to 2 (g/L), L-arginine: 5 to 15 (g/L), L-histidine: 2 to 10 (g/L), L-alanine: 5 to 15 (g/L), L-proline: 2 to 10 (g/L), L-serine: 1 to 7 (g/L), Glycine: 2 to 10 (g/L), L-asparatic acid: 0.2 to 3 (g/L), L-glutamic acid: 0.2 to 3 (g/L).

By adding a small amount of pH adjuster when necessary, the infusion (B) is adjusted to pH 6.5 to 8.0, preferably, to pH 6.7 to 7.5. If pH in the infusion (B) is below 6.5, the mixture of the infusions cannot keep its pH within the optimal range as described later. To the contrary, if pH in the infusion (B) is over 8.0, easily oxidizable amino acids including L-cysteine etc. become more unstable. The both cases are not preferable.

<Vitamin B1>

Vitamin B1 is contained in the infusion (A) as thiamine with 1 to 10 mg/L concentration, preferably, with 2 to 5 mg/L concentration. It is suitable to contain 0.5 to 8 mg of vitamin B1 as absolute amount. As vitamin B1 (thiamine), thiamine hydrochloride, thiamine nitrate, prosultiamine and octotiamine etc. can be used.

<Electrolyte>

(a) Potassium

It is preferable that potassium is contained in the infusion (A) and the infusion (B) separately. As for concentration of potassium contained in each infusion, preferably, the infusion (A) has potassium concentration of 10 to 20 mEq/L and the infusion (B) has potassium concentration of 20 to 40 mEq/L. Potassium contained in the infusion (A) and the infusion (B) is preferably 5 to 30 mEq in total.

As a potassium supply source contained in the infusion (A), potassium chloride, potassium sulfate and the like that are strong electrolytes are preferable. In particular, potassium chloride is more preferable since it is generally used. On the other hand, as a potassium supply source contained in the infusion (B), such compounds as are used in general electrolyte infusions can be used. Potassium chloride, potassium acetate, potassium citrate, potassium dihydrogen phosphate, dipotassium hydrogen phosphate, potassium glycerophosphate, potassium sulfate and potassium lactate can be cited as examples. Among these, phosphate such as potassium dihydrogen phosphate, dipotassium hydrogen phosphate and potassium glycerophosphate is suitable as a phosphorus supply source as well. The potassium supply source may be in hydrated form.

(b) Calcium

It is preferable that calcium is contained only in the infusion (A). If calcium is contained in the infusion (B), precipitation occurs, reacting to phosphate. Prevention of such precipitation is the reason why calcium and phosphate are separated. Calcium chloride that is a strong electrolyte is preferably used as a calcium supply source. It is also preferable that calcium is contained with 2 to 10 mEq/L concentration in the infusion (A).

(c) Sodium

Sodium can be contained in either or both of the infusion (A) and the infusion (B). However, it is preferable to use chloride for potassium and calcium. Therefore, it is preferable that sodium chloride is not used as a sodium supply source to prevent hyperchloremic acidosis from occurring.

When buffering sodium salts such as sodium acetate, sodium citrate, sodium dihydrogen phosphate, disodium hydrogen phosphate and sodium lactate are used, it is preferable that such sodium salts are added to the infusion (B) so as to meet the above-mentioned requirement for titratable acidity in the infusion (A). It is also preferable that sodium is contained with 80 to 150 mEq/L concentration in the infusion (B).

To prevent phosphorus and calcium or magnesium from being precipitated after mixing the infusions, sodium citrate is suitably used as part of sodium supply source.

(d) Other electrolytes

(i) Examples of magnesium supply source include magnesium sulfate, magnesium chloride, magnesium acetate and the like. Among these, magnesium sulfate and magnesium chloride can be contained in the infusion (A) as strong electrolytes.
(ii) Examples of phosphorus supply source include potassium dihydrogen phosphate, dipotassium hydrogen phosphate, potassium glycerophosphate, sodium dihydrogen phosphate, disodium hydrogen phosphate, sodium glycerophosphate and the like. These phosphorus compounds are contained in the infusion (B).
(iii) Examples of zinc supply source include zinc sulfate, zinc chloride and the like. These zinc compounds can be contained in the infusion (A).

As a supply source of each electrolyte mentioned in (i) to (iii), hydrate can be used. However, buffering electrolytes should be added to the infusion (B). Regarding each electrolyte, it is preferable that the infusion (A) contains magnesium with 2 to 10 mEq/L concentration and zinc with approximately 2 to 10 nmol/L concentration. It is also preferable that the infusion (B) contains phosphorus with approximately 10 to 20 mmol/L concentration.

<Additive, Combination Drug>

Additives such as stabilizers can be added to the infusion preparation of the present invention if necessary. However, it is suitable that sulfite such as sodium hydrogen sulfite typical of stabilizers is added to the infusion (B). In addition, if necessary, other combination drugs including each type of vitamins, trace elements (minerals) and the like can be optionally added to and contained in the infusion preparation of the present invention.

<Mixture>

In the infusion preparation of the present invention, the infusion (A) and the infusion (B) are mixed when used. To prevent patients from suffering angialgia and increase safety, the mixture of the infusions (A) and (B) preferably has pH 6 to 7.5 and titratable acidity of 5 to 10. The volume ratio of the infusion (A) and the infusion (B) is preferably 1 to 4:1.

<Infusion Solution Vessel>

A vessel for putting the infusion preparation of the present invention into is not particularly limited, as long as it has two chambers that can be communicated with each other. As examples of a vessel (infusion bag) having two chambers that are separated by an openable partition, the followings can be cited: the vessel which has a partition formed with an easily removable seal (Japanese Patent Publication Laid-Open No. 2-4671, Japanese Utility Model Publication Laid-Open No. 5-5138 etc.), the vessel which has a partition formed by placing a clip between the chambers (Japanese Patent Publication Laid-Open No. 63-309263 etc.), and the vessel which has various openable means prepared for a partition. (Japanese Examined Patent Application Publication No. 63-20550 etc.). Among these, an infusion bag that has a partition formed with an easily removable seal is suitable for mass production and preferable in terms of easy communication.

Examples of materials of the above infusion bag include flexible plastics such as various gas-permeable plastics including polyethylene, polypropylene, polyvinylchloride, cross-linked ethylene-vinyl acetate copolymer, ethylene-alpha-olefin copolymer, and a blend or a laminated body of these respective polymers, each of which is widely used for medical packaging.

The infusion preparation of the present invention can be put into or fill an infusion bag by conventional methods. For example, there is a method of filling each chamber with each infusion in an inert gas atmosphere, then putting a stopper and carrying out heat sterilization. Well-known methods such as high-pressure steam sterilization and hot water shower sterilization can be applied for the heat sterilization. If necessary, heat sterilization can be carried out in an inert gas atmosphere such as carbon dioxide and nitrogen.

Moreover, to surely protect the infusion preparation put into the above infusion bag against deterioration and oxidation, preferably, the infusion bag and a deoxidizer are packed together with an oxygen-barrier outer packaging bag. In particular, when a two-chambered infusion bag that has a partition formed with an easily removable seal is used, it is preferable that the infusion bag is packed, being folded at the portion of an easily removable seal, for example, being folded in two at the portion of an easily removable seal, so that the partition cannot be open due to outside pressure. In addition, if necessary, inert gas filled packaging and the like can be applied.

As materials of a gas-impermeable outer packaging vessel suitable for the above packaging, films and sheets that are made of various materials and generally used can be employed. Specifically, ethylene-vinylalcohol copolymer, polyvinylidene chloride, polyacrylonitrile, polyvinyl alcohol, polyamide, polyester etc. or a material containing at least one of these can be cited as examples.

Regarding a deoxidizer, each type of well-known deoxidizers, specifically, deoxidizers containing iron compounds such as iron hydroxide, iron oxide and iron carbide as an active ingredient or deoxidizers containing low-molecular phenol and active carbon can be used. As typical commercial items, “Ageless” (by Mitsubishi Gas Chemical Co.), “Modulan” (by Nippon Kayaku Co.), “Secule” (by Nippon Soda Co.) and “Tamotsu” (by Oji Kako Co.) can be named.

The present invention will be described in detail below, referring to examples and comparative examples. It is understood, however, that the present invention is not to be regarded as limited to the following examples.

### EXAMPLE 1

<Infusion (A)>

Glucose and each strong electrolyte were dissolved in distilled water for injection with the following concentration to prepare the infusion (A) having the composition as mentioned below. In this infusion (A), a small amount of hydrochloric acid was added to adjust pH to 4.5. The titratable acidity of the infusion (A) was 0.08.

| | | |
|---|---|---|
| Glucose | 107.14 | g/L |
| Potassium chloride | 0.92 | g/L |
| Calcium chloride (2H₂O) | 0.53 | g/L |
| Magnesium sulfate (7H₂O) | 0.88 | g/L |
| Zinc sulfate | 2.00 | mg/L |
| Thiamine hydrochloride | 2.71 | mg/L |

<Infusion (B)>

The following crystalline amino acids and each electrolyte were dissolved in distilled water for injection to prepare the infusion (B) having the composition as mentioned below. In this infusion (B), acetic acid was used as pH adjuster to adjust pH to 6.8.

| | |
|---|---|
| L-leucine | 14.0 g/L |
| L-isoleucine | 8.0 g/L |
| L-valine | 8.0 g/L |
| L-lysine hydrochloride | 13.1 g/L |
| L-threonine | 5.7 g/L |
| L-tryptophan | 2.0 g/L |
| L-methionine | 3.9 g/L |
| L-phenylalanine | 7.0 g/L |
| N-acetyl-L-cysteine | 1.3 g/L |
| L-tyrosine | 0.5 g/L |
| L-arginine | 10.5 g/L |
| L-histidine | 5.0 g/L |
| L-alanine | 8.0 g/L |
| L-proline | 5.0 g/L |
| L-serine | 3.0 g/L |
| Glycine | 5.9 g/L |
| L-asparatic acid | 1.0 g/L |
| L-glutamic acid | 1.0 g/L |
| Dipotassium hydrogen phosphate | 3.31 g/L |
| Disodium hydrogen phosphate | 5.13 g/L |
| Sodium lactate | 7.63 g/L |
| Sodium citrate | 1.77 g/L |
| Sodium hydrogen sulfite | 0.05 g/L |

<Infusion Preparation>

The both infusions obtained as above went through aseptic filtration. The infusion (A) 700 mL and the infusion (B) 300 mL were respectively put into each chamber of a polyethylene vessel that has two chambers separated by an easily removable seal. The infusion (B) went through nitrogen replacement and was closely sealed. Then, high-pressure steam sterilization was performed according to a conventional method. Subsequently, the vessel was folded at the portion of an easily removable seal and was put into an outer packaging bag (oxygen-barrier outer packaging bag) made of multilayer barrier film (Product name: “Bovlon” by NSR), together with a deoxidizer (Product name: “Ageless” by Mitsubishi Gas Chemical Co.). Thereby, the infusion preparation of the present invention was obtained.

After mixing the two infusions constituting this infusion preparation, the mixture had pH 6.7 and titratable acidity of 7.

### EXAMPLE 2

Except that instead of hydrochloric acid in the infusion (A) of Example 1, acetic acid was used to adjust pH to 4.5, an infusion preparation was obtained in a similar way to Example 1. The titratable acidity of the infusion (A) was 0.1, and the concentration of acetic acid was 0.2 mEq/L. After mixing two infusions, the mixture had pH 6.7 and titratable acidity of 7.

### EXAMPLE 3

Except that instead of potassium chloride in the infusion (A) of Example 1, 1.68 g/L of potassium dihydrogen phosphate was added, an infusion preparation was obtained in a similar way to Example 1. The titratable acidity of the infusion (A) was 1. After mixing two infusions, the mixture had pH 6.7 and titratable acidity of 7.

### EXAMPLE 4

Except that instead of 0.88 g/L of magnesium sulfate (7H₂O) in the infusion (A) of Example 2, 0.44 g/L of magnesium sulfate (7H₂O) and 0.38 g/L of magnesium acetate (4H₂O) were added, an infusion preparation was obtained in a similar way to Example 2. The titratable acidity of the infusion (A) was 0.5, and the concentration of acetic acid was 4.4 mEq/L. After mixing two infusions, the mixture had pH 6.6 and titratable acidity of 7.5.

### COMPARATIVE EXAMPLE 1

Instead of 7.63 g/L of sodium lactate added in the infusion (B) of Example 1, 3.27 g/L of sodium lactate was added to the infusion (A). Except for this, an infusion preparation was obtained in a similar way to Example 1. The titratable acidity of the infusion (A) was 4.2.

### COMPARATIVE EXAMPLE 2

Instead of 0.53 g/L of calcium chloride (2H₂O) in the infusion (A) of Example 1, 1.6 g/L of calcium gluconate was added, and instead of 7.63 g/L of sodium lactate added in the infusion (B), 3.27 g/L of sodium lactate was added to the infusion (A). Except for these, an infusion preparation was obtained in a similar way to Example 1. The titratable acidity of the infusion (A) was 4.9.

### COMPARATIVE EXAMPLE 3

Except that instead of magnesium sulfate (7H₂O) in the infusion (A) of Example 2, 0.77 g/L of magnesium acetate (4H₂O) was added, an infusion preparation was obtained in a similar way to Example 2. The titratable acidity of the infusion (A) was 1.6, and the concentration of acetic acid was 8.5 mEq/L. After mixing two infusions, the mixture had pH 6.6 and titratable acidity of 8.

After storing the infusion preparations obtained in the above examples 1 to 4 and comparative examples 1 to 3 under the condition of 60° C. and 75% RH for 14 days, the infusion bags were picked up from the outer packaging bags. The infusion (A) only was withdrawn into a syringe to measure residual volume of vitamin B1 with a high-performance liquid chromatography and figure out the residual ratio of vitamin B1 to initially supplied amount.

TABLE 1

| | | | | | Comparative | Comparative | Comparative |
|---|---|---|---|---|---|---|---|
| | Example 1 | Example 2 | Example 3 | Example 4 | Example 1 | Example 2 | Example 3 |
| Vitamin B1 | 91.3 | 91 | 88.5 | 88 | 80.3 | 75.5 | 82.5 |
| residual ratio | | | | | | | |
| (%) | | | | | | | |

It is apparent from the result shown in Table 1 that compared to the infusion preparations of comparative examples 1 to 3, the infusion preparations of examples 1 to 4 have more stable vitamin B1 contained in the infusion (A).

1. An infusion preparation for peripheral parenteral administration, comprising an infusion (A) containing glucose with 80 to 200 g/L concentration and an infusion (B) containing amino acid with 50 to 150 g/L concentration that are separately put into a vessel that is divided with an openable partition means;
wherein the infusion (A) does not contain sulfite, has titratable acidity of 1 or less, contains vitamin B1 and is adjusted to pH 3 to 5;
the infusion (B) is adjusted to pH 6.5 to 8; and
after mixing the infusion (A) and the infusion (B), the mixture has pH 6 to 7.5 and titratable acidity of 5 to 10.

2. The infusion preparation for peripheral parenteral administration according to claim 1, wherein the infusion (A) contains carboxylic acid and its salt with 0 to 5 mEq/L concentration.

3. The infusion preparation for peripheral parenteral administration according to claim 1, wherein electrolytes contained in the infusion (A) are all strong electrolytes.

4. The infusion preparation for peripheral parenteral administration according to claim 3, wherein the infusion (A) has electrolyte composition of K⁺: 10 to 20 mEq/L, Ca²⁺: 2 to 10 mEq/L, Mg²⁺: 2 to 10 mEq/L, Cl⁻: 12 to 30 mEq/L, Zn: 2 to 10 mmol/L; the infusion (B) has electrolyte composition of Na⁺: 80 to 150 mEq/L, K⁺: 20 to 40 mEq/L, P: 10 to 20 mmol/L; and the volume ratio (A:B) of the infusion (A) and the infusion (B) is 1 to 4:1.

5. The infusion preparation for peripheral parenteral administration according to claim 4, wherein calcium and potassium are contained respectively in the form of chloride as the strong electrolytes; and sodium chloride is not used as a sodium supply source in either of the infusions (A) or (B).

6. The infusion preparation for peripheral parenteral administration according to claim 1, wherein amino acid composition in the infusion (B) is shown as follows in terms of free amino acid: L-leucine: 10 to 20 (g/L), L-isoleucine: 5 to 15 (g/L), L-valine: 5 to 15 (g/L), L-lysine: 5 to 15 (g/L), L-threonine: 2 to 10 (g/L), L-tryptophan: 0.5 to 5 (g/L), L-methionine: 1 to 8 (g/L), L-phenylalanine: 3 to 15 (g/L), L-cysteine: 0.1 to 3 (g/L), L-tyrosine: 0.1 to 2 (g/L), L-arginine: 5 to 15 (g/L), L-histidine: 2 to 10 (g/L), L-alanine: 5 to 15 (g/L), L-proline: 2 to 10 (g/L), L-serine: 1 to 7 (g/L), Glycine: 2 to 10 (g/L), L-asparatic acid: 0.2 to 3 (g/L), L-glutamic acid: 0.2 to 3 (g/L).

7. The infusion preparation for peripheral parenteral administration according to claim 6, wherein L-cysteine is contained as N-acetyl compound.

8. The infusion preparation for peripheral parenteral administration according to claim 7, wherein vitamin B1 is contained in the infusion (A) with 1 to 10 mg/L concentration in terms of thiamine.

9. The infusion preparation for peripheral parenteral administration according to claim 1, wherein the vessel is a flexible plastic infusion bag having at least two chambers that are separated by an easily-removable seal.

10. The infusion preparation for peripheral parenteral administration according to claim 9, wherein the infusion bag was folded at the portion of the easily removable seal and was put into an oxygen-barrier outer packaging bag, together with a deoxidizer.

11. The infusion preparation for peripheral parenteral administration according to claim 1, which goes through heat sterilization.

12. A method for stabilizing vitamin B1 in an infusion preparation for peripheral parenteral administration which comprises an infusion (A) containing vitamin B1 and 80 to 200 g/L of glucose and an infusion (B) containing 50 to 150 g/L of amino acid and being adjusted to pH 6.5 to 8 that are separately put into a vessel that is divided with an openable partition means and wherein after mixing the infusion (A) and the infusion (B), the mixture has pH 6 to 7.5 and titratable acidity of 5 to 10,
wherein the infusion (A) does not contain sulfite, has titratable acidity of 1 or less and is adjusted to pH 3 to 5.

13. The method for stabilizing vitamin B1 according to claim 12, wherein the infusion (A) contains carboxylic acid and its salt with 0 to 5 mEq/L concentration.

14. The method for stabilizing vitamin B1 according to claim 12, wherein electrolytes contained in the infusion (A) are all strong electrolytes.
